(19) United States
(12) Patent Application Publication
(10) Pub. No.: US 2009/0161068 A1
(43) Pub. Date: Jun. 25, 2009

(54) Ophthalmic Measurement Apparatus

(76) Inventors: Ming Lai, Webster, NY (US); Barry T. Eagan, Spencerport, NY (US); Daozhi Wang, Rochester, NY (US)

Correspondence Address: Bausch & Lomb Incorporated, One Bausch & Lomb Place, Rochester, NY, 14604-2701, US

(21) Appl. No.: 11/961,409

(22) Filed: Dec. 20, 2007

(51) Int. Cl. A61B 3/15

(52) U.S. Cl. 351/208; 351/206

(57) ABSTRACT

An ophthalmic measurement apparatus having an instrument axis and comprising (A) a central housing comprising at least two reference surfaces and comprising a beam splitter, the instrument axis extending from the central housing, (B) a camera subsystem having a reference surface, the camera subsystem coupled to one of said reference surfaces such that the reference surface to which the subsystem is coupled and the camera subsystem reference surface together operatively align the camera with the instrument axis, and (C) an aberrometer subsystem having a reference surface, the aberrometer subsystem coupled to one of said reference surfaces such that the reference surface to which the aberrometer subsystem is coupled and the reference surface of the aberrometer subsystem together operatively align the aberrometer subsystem with the instrument axis.

### FIELD OF INVENTION

The present invention relates to ophthalmic measurement apparatus, and more particularly to multifunctional ophthalmic measurement apparatus.

### BACKGROUND OF THE INVENTION

Ophthalmologists and optometrists would like to have an accurate representation of subjects' eye performance and physical structure. This information may be used to prescribe corrective lenses (e.g., spectacles, intraocular lenses, corneal implants), to reshape corneas by surgical procedures, and to otherwise treat eye abnormalities. As eye treatments and diagnoses become more complicated, more types of eye measurements techniques (e.g., topography, aberrometry, pachymetry) are being used. Since different types of measurements typically require different types of instruments, a subject is frequently presented to multiple instruments each capable of performing one or more measurement techniques.

Instruments for eye measurement include, for example, aberrometers (for measuring a wavefront produced by an eye), pachymeters (for measuring thicknesses of features of an eye), and topographers (for measuring surface contour of an eye), and axial length measurement instruments. Since measurements are typically made without contacting the eye, remote sensing measurement techniques are used to produce these data. Such techniques typically involve projecting light onto a subject's eye and receiving reflected and/or scattered light with a light detector (e.g., a camera).

To avoid complication of adding additional instruments to a single apparatus, it has been common that a subject, who is to be measured by two or more of the above instruments, be positioned in front of a first instrument for measurement using one or more techniques (e.g., aberrometry) and subsequently, to perform another technique, moved to another location or otherwise oriented such that the subject is positioned in front of another instrument (e.g., a pachymeter). Movement from one location to another is inconvenient for the subject and time consuming for medical staff because it requires alignment of the subject to multiple apparatus and may require multiple data entry steps for patient identification.

While some apparatus have been constructed that are capable of performing multiple measurement techniques, such instruments have been limited in their capability. The ability to provide multifunctionality in a single apparatus has been limited by complexity of manufacturing and servicing such apparatus. For example, such apparatus have been constructed with components of each of the instruments on a single mounting board with discrete components of the instruments being aligned to achieve an overall alignment of the instrument. Frequently, the alignment is such that there is an interdependence of the alignment of the components of one instrument with the components of another instrument. Such an arrangements have made manufacture of multifunctional apparatus difficult, and has made customization and servicing of such apparatus, in the field, difficult.

### SUMMARY

Aspects of the present invention are directed to an ophthalmic measurement apparatus having an instrument axis and comprising (A) a central housing comprising at least two reference surfaces and comprising a beam splitter, the instrument axis extending from the central housing, (B) a camera subsystem having a reference surface, the camera subsystem coupled to one of said reference surfaces such that the reference surface to which the subsystem is coupled and the camera subsystem reference surface together operatively align the camera with the instrument axis, and (C) an aberrometer subsystem having a reference surface, the aberrometer subsystem coupled to one of said reference surfaces such that the reference surface to which the aberrometer subsystem is coupled and the reference surface of the aberrometer subsystem together operatively align the aberrometer subsystem with the instrument axis.

In some embodiments, the central housing comprises a third reference surface, and the apparatus further comprises (D) a third ophthalmic subsystem having a reference surface, the third subsystem being coupled to the third reference surface such that the third reference surface and the reference surface of the third subsystem together operatively align the third subsystem with the instrument axis.

In some embodiments, each reference surface forms a portion of a different wall of the central housing. In some embodiments, at least two of the walls are perpendicular to one another. In some embodiments, at least two of the walls are integrally formed.

The third ophthalmic subsystem may comprise a Placido disk.

In some embodiments, at least one of the subsystems is directly connected to a corresponding reference surface. In some embodiments, the aberrometer subsystem is adapted to project light onto a subject's eye. In some embodiments, the aberrometer subsystem is adapted to image light scattered from a subject's eye.

In some embodiments, the third subsystem is adapted to facilitate at least one of topographic measurement, pachymetric measurement or axial length measurement of an eye.

In some embodiments, at least one of the reference surfaces comprises at least two discrete segments. In some embodiments, at least one of the reference surfaces comprises threading. At least one of the reference surfaces may comprise threading and a side pin.

Another aspect of the invention is directed to an ophthalmic measurement apparatus having an instrument axis and comprising (A) a central housing comprising a beam splitter, the instrument axis extending from the central housing, (B) a camera subsystem, (C) means to operatively align the camera with the instrument axis, (D) an aberrometer subsystem; and (E) means to operatively align the aberrometer subsystem with the instrument axis.

Yet another aspect of the invention is directed to an ophthalmic measurement apparatus kit having an instrument axis and comprising (A) a central housing comprising at least two reference surfaces and comprising a beam splitter, the instrument axis extending from the central housing, (B) a camera subsystem having a reference surface, the camera subsystem adapted to be coupled to one of said reference surfaces such that the reference surface to which the subsystem is coupled and the camera subsystem reference surface together operatively align the camera with the instrument axis, and (C) an aberrometer subsystem having a reference surface, the aberrometer subsystem adapted to be coupled to one of said reference surfaces such that the reference surface to which the aberrometer subsystem is coupled and the reference surface of the aberrometer subsystem together operatively align the aberrometer subsystem with the instrument axis.

The term “housing” as used herein refers to a structure having at least three surfaces at least partially bounding a space on at least three mutually normnal directions. In some embodiments all of the surfaces are mechanical reference surfaces. In some embodiments, each of the surfaces is formed on a portion of a different wall. In some embodiments, the structure comprises at least four surfaces or at least five surfaces. A reference surface may comprise a portion of a side of a wall or may form an entire side.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of an example of an ophthalmic measurement apparatus according to aspects of the present invention;

FIG. 2 is a schematic illustration of an example of a central housing alone (i.e., subsystems are removed);

FIG. 3A is a top view, schematic illustration of another embodiment of an apparatus according to aspects of the present invention;

FIG. 3B is a side view, schematic illustration of the apparatus of FIG. 3A;

FIG. 4 illustrates a central housing having curved mechanical reference surfaces;

FIG. 5A is a partial view of an apparatus in which a mechanical reference surface and a subsystem reference surface are coupled together and pins operate to limit translational movement as well as angular movement;

FIG. 5B illustrates a reference surface having discrete segments;

FIG. 5C is a partial view of an apparatus comprising a mechanical reference surface having threading;

FIG. 5D is a partial view of an apparatus comprising a mechanical reference surface including a side pin and threading; and

FIG. 5E is a view of the apparatus of FIG. 5D along lines 5E-5E.

### DETAILED DESCRIPTION

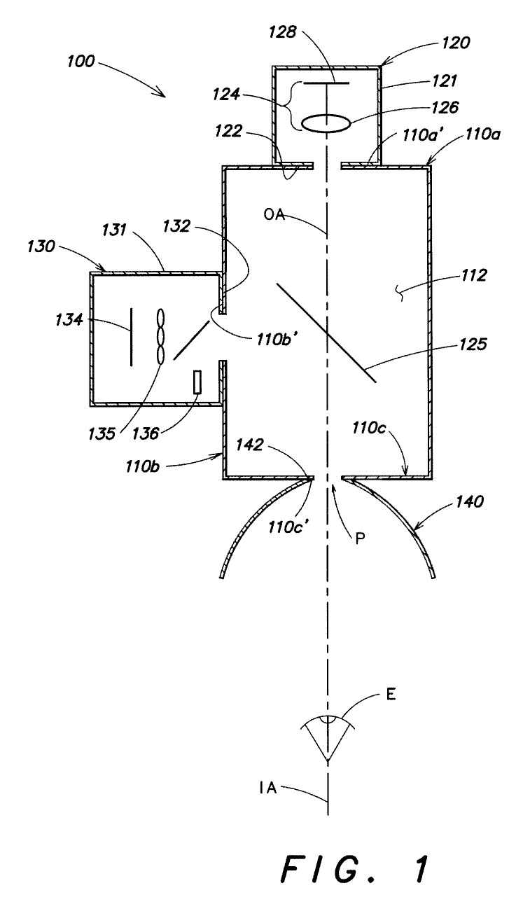

FIG. 1A is a schematic illustration of an example of an ophthalmic measurement apparatus 100 according to aspects of the present invention. The instrument is capable of performing a plurality of measurement techniques. The apparatus has an instrument axis IA (i.e., an axis with which a subject's eye E is aligned to make multiple measurements). The apparatus comprises a central housing 100, and three subsystems 120, 130, 140.

Central housing 110 has instrument axis IA extending therefrom. It will be appreciated that, according to aspects of the present invention, a subject can be aligned with the instruments axis, preferably a single time, and measurements using multiple techniques can be performed on the subject.

The central housing contains a beam splitter 125, which permits optical elements of one or more of the subsystems to be connected to the central housing in a manner such that an optical axis of an instrument is operatively aligned with the instrument axis of the apparatus.

Central housing 110 comprises three mechanical reference surfaces that at least partially bound space 112 occupied by the beam splitter, and provide a path through which light passes along the instrument axis between eye E and each of the subsystems. In the illustrated embodiment, each reference surface 110a′, 110b′, 110c′ constitutes a portion of a corresponding wall 110a, 110b and 110c.

Each of the subsystems has a corresponding subsystem reference surface 122, 132, 142 that interfaces with a mechanical surface 110a′, 110b′, 110c′ of the central housing to provide alignment with the instrument axis. It will be understood that one or more subsystems may project and/or receive light directly along the instrument axis (i.e., without redirection by beam splitter or any other steering optics). Subsystem 120 is an example of such a subsystem. It will also be understood that one or more subsystems may project light along the instrument axis when connected to the central housing only after the light is incident on steering optics (e.g., beam splitter 125). Subsystem 130 is an example of such a subsystem. For example, the steering optics may include one or more beam splitters (e.g., beam splitter 125) and possibly one or more mirrors (not shown).

First subsystem 120 comprises a camera 124 and a housing 121 having a reference surface 122. The camera subsystem is coupled to wall 110a of the central housing such that a mechanical reference surface 110a′ and surface 122 together operatively align camera 124 with the instrument axis IA. Camera 124 comprises a lens system 126 and sensor 128 that are adapted to receive light from an eye E and form an image of the eye. Camera 124 receives light directly along camera optical axis OA and instrument axis IA. It will be appreciated that the optical axis OA has a predetermined angular and translational relationship with subsystem reference surface 122 such that, when subsystem 120 and reference surface 122 are coupled together, the optical axis aligns with the instrument axis. As one of ordinary skill in the art would understand, a “mechanical reference surface” (or simply a “reference surface”) is a precisely manufactured (e.g., machined) surface suitable to achieve precise mechanical positioning. Typically, a pair of mechanical reference surfaces (e.g., one on a central housing and one on a subsystem) achieves alignment and one or more dwell pins can be used to facilitate and maintain alignment. By this technique, mechanical positioning between a pair of reference surfaces can achieve and maintain translational alignment within 100 microns (i.e., ±50 microns), and preferably within 25 microns (i.e., ±12.5 microns); and angular alignment within ±3 milliradians, and preferably better than ±1 milliradian, can also be achieved. Screws may be added to make alignment more permanent.

For example, camera 124 may be what is commonly referred to as a pupil camera. Such cameras may be used to measure eye dimension; however, as described below, one or more subsystems may use the camera to perform one or more measurement techniques.

Aberrometer subsystem 130 comprises first components (e.g., lenslet array 135, light sensor 134 and laser 136) adapted to facilitate measurement of aberrations of eye E and a housing 131 having a subsystem reference surface 132. The aberrometer subsystem is coupled to wall 110b such that a mechanical reference surface 110b′ of the wall and surface 132 of the subsystem operatively align the first components with the instrument axis (i.e., the first components have a predetermined relationship with the instrument axis so as to facilitate aberration measurement). The first components are adapted to project illumination light (e.g., a beam of light) onto the eye and to form an image of the illumination light after it has been scattered from the eye. It will be appreciated that a beam from laser 136 and an optical axis of the camera (which comprises lenslet array 135 and sensor 134) have a predetermined angular and translational relationship with surface 132 such that when subsystem 130 and reference surface 110b′ are coupled together, the camera and the beam from laser 136 are operatively aligned with the instrument axis IA.

In the illustrated embodiments, aberrometer subsystem comprises a Hartmann Shack device. However, any suitable aberrometer device may be used.

In some embodiments, the aberrometer subsystem is adapted to perform only one of projection of illumination light and formation of an image using illumination light after it impinges on the eye. In such embodiments, a second aberrometer subsystem may be included to perform the other project illumination or formation of an image. The second aberrometer subsystem may be coupled to one of said walls such that a reference surface of the wall to which the aberrometer subsystem is connected and the reference surface of the second aberrometer subsystem operatively align the components associated therewith with the instrument axis; however, such an arrangement is not necessary.

A third ophthalmic subsystem 140 comprises optical components and a reference surface 142. Third subsystem 140 is coupled to one of said walls 110c such that a reference surface 110c′ of the wall and surface 142 together operatively align the third subsystem with instrument axis IA. In the illustrated embodiment, subsystem 140 includes a conventional Placido disk including a plurality of ring illumination sources (not shown). It will be appreciated that light from the source is operatively aligned with the instrument axis IA. Camera 124 is used to capture images of the eye generated using illumination from the rings.

In some embodiments, a fourth optical subsystem comprising optical components and a reference surface that, together with a mechanical reference surface of the central housing, operatively align the fourth subsystem with the instrument axis IA. For example, the optical components of the fourth subsystem may facilitate one of aberrometry, pachymetry, topography or axial length measurement. The fourth subsystem may include illumination optics to facilitate one of the above eye measurement techniques and/or receive optics (e.g., including a light detector) to facilitate one of the above eye measurement techniques.

It will be appreciated that operative alignment, as used herein, does not require alignment of a subsystem such that a subsystem axis is coincident along the instrument axis. That is operative alignment may occur when the subsystem axis has an angular and/or a translational displacement from the instrument axis (i.e., some instruments are operatively aligned in an off-axis location). For example, alignment need not result in an optical axis or a beam of a subsystem coincident along the instrument axis.

Coupling between a subsystem and a mechanical reference surface can be achieved in any manner that achieves the operative alignment determined by an identified reference surface of the subsystem and a mechanical reference surface of the housing. For example, for a given subsystem, the reference surface and the subsystem reference surface can form a direct connection by direct contact of the reference surface and the subsystem reference surface. In other embodiments, an intervening connection element can be used provided that the operative alignment is achieved.

It will be appreciated that coupling apparatus used to couple a housing to the central housing preferably achieves and maintains structural integrity of instrument such that operative alignment is maintained. Failure to maintain such integrity may result in inaccurate measurements and inability to accurately align measurement outputs from the first instrument and the second instrument relative to one another. For example, the housing and reference surfaces may be made of aluminum or stainless steel, and coupling apparatus (e.g., bolts, screws, pins or other apparatus) that is used to maintain coupling may be made of aluminum or stainless steel.

Although the illustrated embodiment includes three subsystems coupled to the central housing, embodiments of the present invention may include two or more subsystems so connected. More than one subsystem can be connected to a given reference surface. More than one subsystem can be connected to a given wall. Multiple reference surfaces can be formed on a given wall.

It will be appreciated that an instrument constructed according to aspects of the present invention comprises a central housing having subsystems coupled thereto to facilitate manufacture and servicing of instruments. For example, optical components within a given subsystem can be operatively aligned with the instrument axis by relatively simple connection of a reference surface of the subsystem with a reference surface of the central housing. Preferably, once attachment is achieved, no further alignment of the instrument is needed to achieve operative alignment of the optical components with the instrument axis.

In some embodiments, the apparatus as described above may be provided as an unassembled kit. Accordingly, the kit comprises a central housing and one or more of a camera subsystem as described above, an aberrometer subsystem as described above or another system that are not coupled to a mechanical reference surface of the central housing as set forth above. At least one of the uncoupled subsystems has a reference surface (and associated coupling apparatus) adapted such that the subsystem reference surface and a mechanical reference surface of the central housing together operatively align the one or more subsystems with the instrument axis and appropriately maintain the alignment.

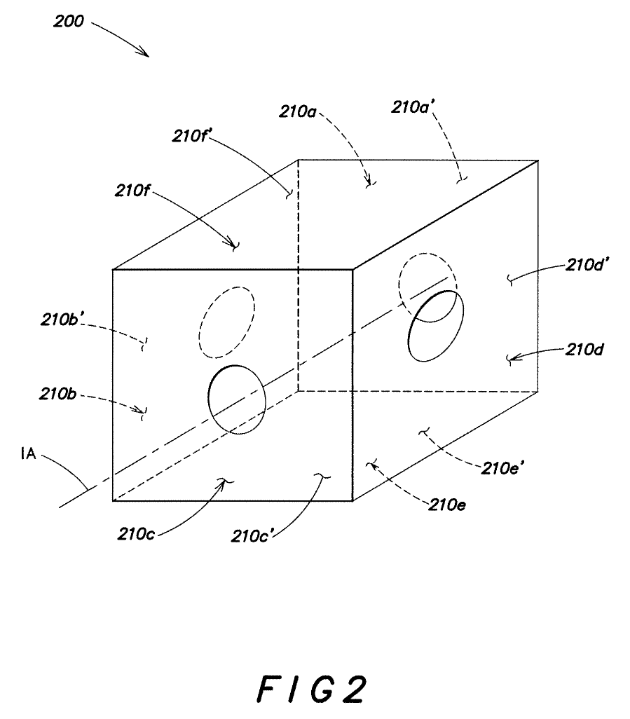

FIG. 2 is a schematic illustration of an example of a central housing 200 alone (i.e., all subsystems are removed). The housing comprises six walls 210a-210f. The walls are connected together perpendicular to one another with ports extending through some of the walls (including through corresponding mechanical reference surfaces 210a′-210f′) to permit light to be transmitted through the wall and reference surface. Subsystems can be connected to a corresponding reference surface with the port and aligned to permit projection and/or receipt of light therethrough by a subsystem. It will be appreciated that, according to aspects of the invention, the walls need not be perpendicular to one another. In some embodiments, one or more of the wall may be integrally formed with one or more other walls. Also, the mechanical reference surfaces need not be perpendicular to one another. In some embodiments, one or more of the mechanical reference surfaces may be integrally formed with one or more other mechanical reference surfaces.

Typically, optical components (e.g., beam splitters) which are not shown will be mounted on a first of the walls (e.g. 210c) of the central housing, a first subsystem will be coupled to a reference surface 210a′ of a second of the walls (e.g., 210a), and a second subsystem will be coupled to a reference surface 210b′ of a third of the walls (e.g., 210b).

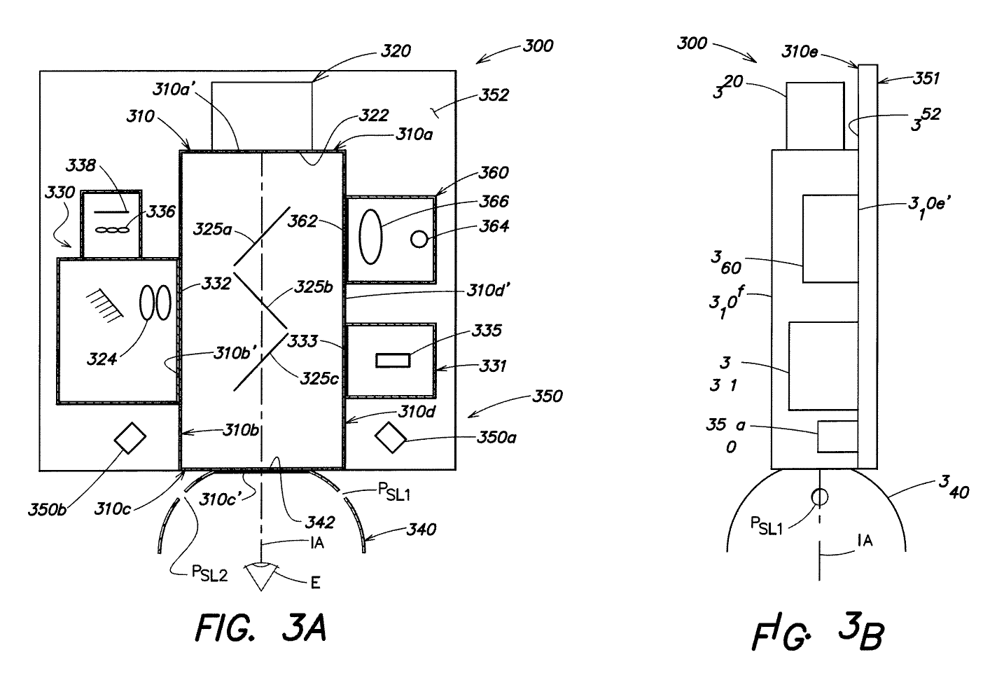

FIGS. 3A and 3B, respectively, are top view and side view, schematic illustrations of another embodiment of an apparatus 300 according to aspects of the present invention. A subject's eye E is disposed in front of the apparatus.

The apparatus comprises a central housing 310 having six walls 310a-310f. Three beam splitters 325a-325c are disposed in the housing connected to a reference surface 310e′. The beam splitters direct light between instrument axis IA and a corresponding one of subsystems 330, 331 and 360 through appropriate ports (not shown).

The apparatus comprises a pupil camera subsystem 320 having a reference surface 322. The camera subsystem is connected to one of said walls 310a such that a mechanical reference surface 310a′ of wall 310a and surface 322 together operatively align the pupil camera with the instrument axis IA.

The apparatus comprises a Placido topographer subsystem 340 comprising a reference surface 342 connected to one of said walls 310c such that a mechanical reference surface 310c′ of the wall and surface 342 of the subsystem 340 together operatively align subsystem 340 with the instrument axis.

The apparatus comprises a first aberrometer subsystem 330 comprising a relay lens 334, a lenslet array 336 and a detector 338. A reference surface 332 of subsystem 330 is connected to one of said walls 310b such that a mechanical reference surface 310b′ of the wall and the surface 332 together operatively align the relay lens, the lenslet array and detector with the instrument axis.

The apparatus comprises a second aberrometer subsystem 331 comprising an injection laser 335 and a reference surface 333 connected to one of said walls 310d such that a mechanical reference surface 310d′ of the wall and the surface 333 together operatively align the injection laser with the instrument axis.

The apparatus comprises a pachymeter illumination subsystem 350 comprising two slit projectors 350a, 350b and a platform 351 comprising a reference surface 352 connected to one of said walls 310e (shown in FIG. 3B) such that a mechanical reference surface 310e′ of the wall and surface 352 together operatively align the ophthalmic projector such that slits of light are suitably projected onto eye E. Slits of light are projected through ports P_{SL1} and P_{SL2} in subsystem 340. It will be appreciated that the pupil camera in subsystem 320 is used as a detector with both the Placido topographer subsystem and the pachymeter subsystem.

The apparatus also comprises a fixation subsystem 360 comprising a fixation target 364 and lens 366 through which a subject views the target. Subsystem 360 comprises a reference surface 362 connected to walls 310d such that mechanical reference surface 310d′ of the wall and surface 362 together operatively align the target with eye E. In the illustrated embodiment, wall 310f operates as a cover for the central housing to protect any components in the housing from damage or debris.

It will be appreciated that, according to aspects of the present invention, a subject's eye E can be aligned with the instruments axis IA a single time, and topographic, aberrometric, pachymetric measurements can performed on the subject.

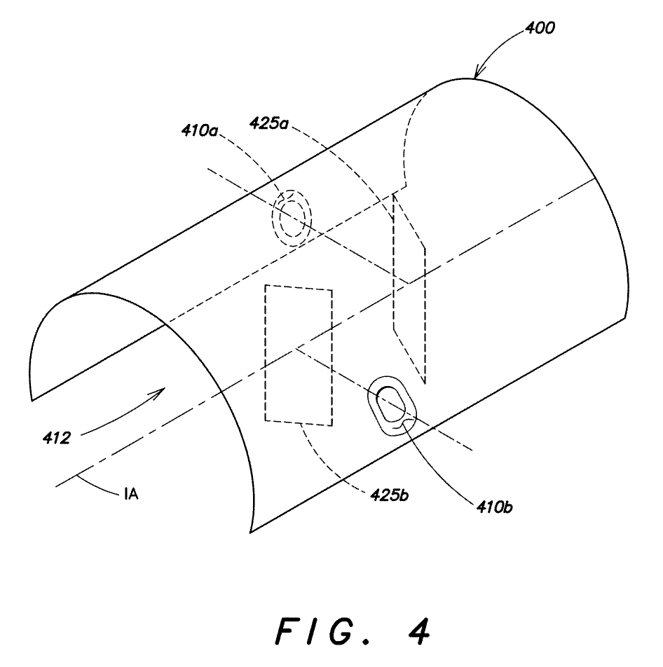

Although, in some embodiments, the mechanical reference surfaces and the subsystem reference surfaces are illustrated as flat surfaces, in other embodiments, the mechanical reference surface and/or the subsystem reference surface may be curved or angulated provided that, together, they operatively align a given subsystem with the instrument axis. FIG. 4 illustrates a central housing 400 having curved mechanical reference surfaces 410a, 410b that at least partially bounds space 412 occupied by the beam splitters 425a and 425b.

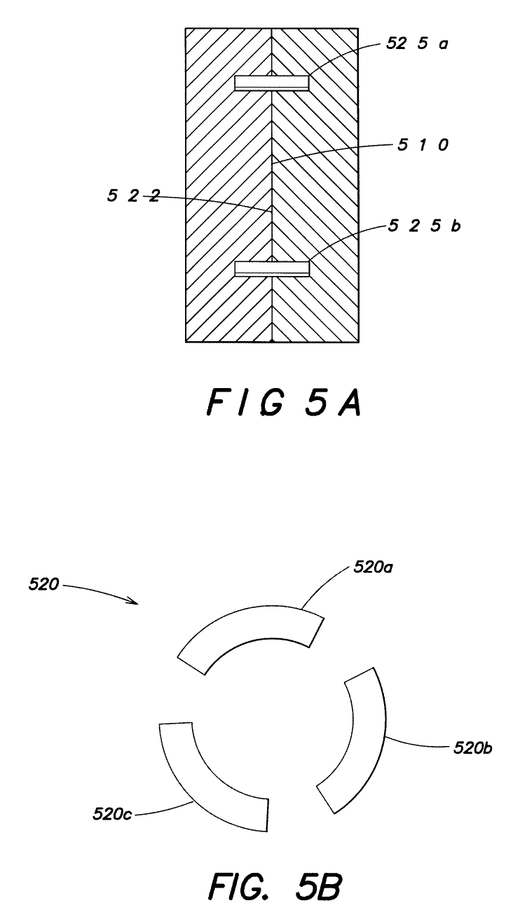

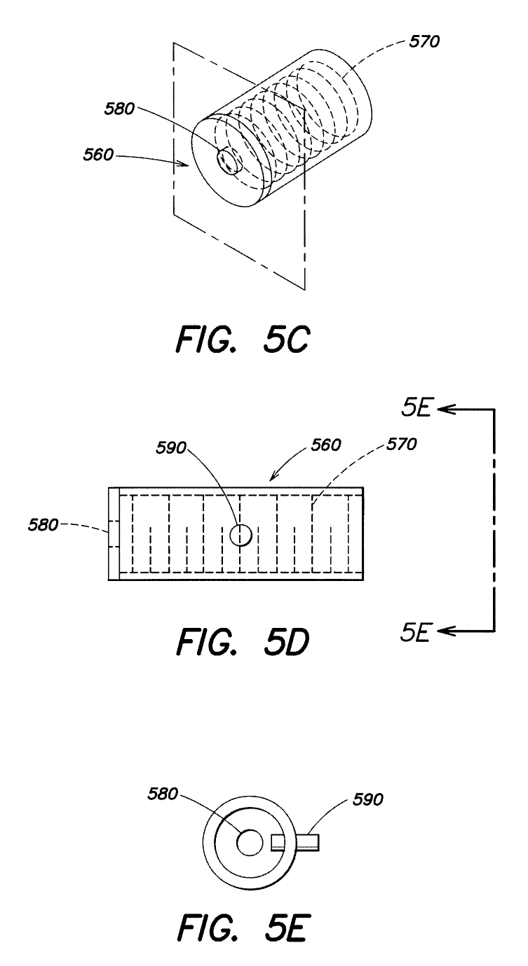

FIG. 5A is a partial view of an embodiment of an apparatus that illustrates a mechanical reference surface 510 and a subsystem reference surface 522 coupled together and pins 525a and 525b (i.e., coupling apparatus) operate to limit translational movement as well as angular movement of the subsystem. In some embodiments, the use of two coupling apparatus (e.g., pins) is preferable to provide adequate constraint without providing overconstraint. In some embodiments, a single gimbal mount is used to couple a subsystem to the central housing. As shown in FIG. 5B, a mechanical reference surface or subsystem reference surface 510 may be continuous or have discrete segments 510a, 510b and 510c.

FIG. 5C is a partial view of an embodiment of a central housing comprising a mechanical reference surface 560 having threading 570. A port 580 extends through the central housing. A subsystem having threading (not shown) can be screwed into the central housing threading to couple a surface of the subsystem with the reference surface thereby operatively aligning the subsystem with the instrument axis IA. FIG. 5D is a partial view of an embodiment of a central housing comprising a mechanical reference surface 560 comprising a side pin 590 and threading 570. A port 580 extends through the central housing. It will be appreciated that in such an arrangement the side pin provides at least a portion of the reference surface and, in combination with the threading, determines both axial alignment (by limiting travel) and determines angular alignment by limiting rotation of the subassembly.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An ophthalmic measurement apparatus having an instrument axis and comprising:
(A) a central housing comprising at least a first reference surface and a second reference surface and comprising a beam splitter, the instrument axis extending from the central housing;
(B) a camera subsystem comprising a second housing having a camera subsystem reference surface, the camera subsystem coupled to one of said first and second reference surfaces such that the one of said first and second reference surfaces to which the camera subsystem is coupled and the camera subsystem reference surface together operatively align the camera with the instrument axis; and
(C) an aberrometer subsystem comprising a third housing having a aberrometer subsystem reference surface, the aberrometer subsystem coupled to another of said first and second reference surfaces such that the reference surface to which the aberrometer subsystem is coupled and the aberrometer reference surface together operatively align the aberrometer subsystem with the instrument axis.

2. The apparatus of claim 1, wherein the central housing comprises a third reference surface, the apparatus further comprising (D) a third ophthalmic subsystem having a third subsystem reference surface, the third subsystem coupled to the third reference surface such that the third reference surface and the third subsystem reference surface together operatively align the third subsystem with the instrument axis.

3. The apparatus of claim 2, wherein each of the first and second reference surfaces forms a portion of a different wall of the central housing.

4. The apparatus of claim 3, wherein at least two of the walls are perpendicular to one another.

5. The apparatus of claim 3, wherein at least two of the walls are integrally formed.

6. The apparatus of claim 2, wherein the third ophthalmic subsystem comprises a Placido disk.

7. The apparatus of claim 1, wherein at least one of the subsystems is directly connected to a corresponding one of the first reference surface and the second reference surface.

8. The apparatus of claim 1, wherein the aberrometer subsystem is adapted to project light onto a subject's eye.

9. The apparatus of claim 1, wherein the aberrometer subsystem is adapted to image light scattered from a subject's eye.

10. The apparatus of claim 2, wherein the third subsystem is adapted to facilitate at least one of topographic measurement, pachymetric measurement or axial length measurement of an eye.

11. The apparatus of claim 1, wherein at least one of the reference surfaces comprises at least two discrete segments.

12. The apparatus of claim 1, wherein at least one of the first and second reference surfaces comprises threading.

13. The apparatus of claim 1, wherein at least one of the first and second reference surfaces comprises threading and a side pin.

14. An ophthalmic measurement apparatus having an instrument axis and comprising:
(A) a central housing comprising a beam splitter, the instrument axis extending from the central housing;
(B) a camera subsystem
(C) means to operatively align the camera with the instrument axis;
(D) an aberrometer subsystem; and
(E) means to operatively align the aberrometer subsystem with the instrument axis.

15. An ophthalmic measurement apparatus kit having an instrument axis and comprising:
(A) a central housing comprising at least a first reference surface and a second reference surface and comprising a beam splitter, the instrument axis extending from the central housing;
(B) a camera subsystem having a first housing having a first housing reference surface, the camera subsystem adapted to be coupled to one of said first and second reference surfaces such that the reference surface to which the subsystem is coupled and the camera subsystem reference surface together operatively align the camera with the instrument axis; and
(C) an aberrometer subsystem having a second housing having a second housing reference surface, the aberrometer subsystem adapted to be coupled to one of said first and second reference surfaces such that the reference surface to which the aberrometer subsystem is coupled and the aberrometer subsystem reference surface together operatively align the aberrometer subsystem with the instrument axis.
